(19) United States
(12) Patent Application Publication
(10) Pub. No.: US 2008/0281213 A1
(43) Pub. Date: Nov. 13, 2008

(54) CARDIAC COUPLED RESPIRATION COORDINATION SYSTEM

(76) Inventors: Daniel E. Roach, Calgary (CA); Henry J. Duff, Calgary (CA); Robert S. Sheldon, Calgary (CA)

Correspondence Address: Lambert Intellectual Property Law, Suite 200, 10328 - 81 Avenue, Edmonton, AB, T6E 1X2, CA

(21) Appl. No.: 12/122,515

(22) Filed: May 16, 2008

Related U.S. Application Data
(63) Continuation-in-part of application No. 10/846,576, filed on May 17, 2004, now abandoned.

(30) Foreign Application Priority Data
Mar. 8, 2004 (CA) 2,460,039

(51) Int. Cl. A61B 5/02

(52) U.S. Cl. 600/508

(57) ABSTRACT

A series of feed-forward applications are designed to coordinate heart/respiration/muscle and brain to produce a better quality of life endpoint. The applications will be used to treat anxiety, sleep disorders, arrhythmias and enhanced performance during athletic training. The coordination may be used as part of a diagnostic tool.

### CROSS-REFERENCE TO RELATED APPLICATIONS

This application is a continuation-in-part of and claims the priority of an application entitled “CARDIAC COUPLED RESPIRATION COORDINATION SYSTEM”, application Ser. No. 10/846,576 filed May 17, 2004, the content of which is hereby incorporated by reference.

### BACKGROUND

Abnormalities of autonomic nervous system function underlie a number of medical conditions that contribute to substantial morbidity including anxiety, sleep disorders, and arrhythmias. Individuals with anxiety, panic attacks and sleep disorders experience impaired role functioning, spend more days in bed due to illness, have more work days lost, have increased impairment at work, and high use of health services. These problems are common, likely occurring in 2-5% of the general population in North America or Europe. The disability of anxiety is just as great as that caused by other common medical conditions, such as hypertension, diabetes and arthritis. Pharmacologic treatment of these conditions is fraught with side effects, non-compliance and inefficacy.

Improvements in human physiological performance due to coupling of human physiological activities with other human physiological activities is an area of much current study. For example, Kullock, U.S. Pat. No. 6,644,976 issued Nov. 11, 2003, discusses time correlation of movements and physiological components. Kullock requires a synergic programs module that directs physiological movement according to dual correlation factors.

Sudden cardiac death kills approximately 400,000 North Americans annually. Patients with sustained ventricular tachyarrhythmias and severe left ventricular (LV) dysfunction are at high risk of sudden death. As a reaction to the LV dysfunction and manifest arrhythmia, highest risk patients undergo implantation of cardiac defibrillators (ICD). However, most patients otherwise destined to die of arrhythmic death do not have the clinical manifestations of severe LV dysfunction and sustained arrhythmias. Thus, limiting the implantation of ICDs to the group with clinical manifestations of these conditions will fail to prevent the vast majority of cardiac arrests. Accordingly, there is a compelling need to develop easy and widely applicable tools to reliably assess an individual's risk for sudden cardiac death. The superimposition of mild LV dysfunction, altered baroreflex activity, and the presence of T-wave alternans has been found to be a reasonably reliable metric of risk for arrhythmic death. However, vagal-mediated cardiac baroreflex measurements usually require phenylepherine infusions under the guidance of a cardiologist, and the measurement of T-wave alternans requires a noise reduction system to compensate for respiratory induced changes in thoracic electrical impedance.

The autonomic control mechanisms that maintain healthy heart rate, respiratory rate, blood pressure, and O2 saturation are complexly coupled. Despite a high degree of coupling, experts tend to describe specific heart rate changes as they relate to specific changes in other physiological variables. In some embodiments, the heart rate changes caused (either directly or indirectly) by respiration, and the heart rate changes caused by the baroreflex response to blood pressure (BP) changes are of interest. Heart rate changes related to respiration are referred to as respiratory sinus arrhythmia (RSA), and they are often measured by the sum of the power spectral density function (PSD) within the so-called high frequency (HF) band (i.e., 0.15<frequency<0.4 Hz) as derived from the Fourier transform of the sequence of RR-interval (i.e., time between cardiac R-waves) values. Likewise, heart rate changes related to baroreflex activity are often measured by the sum of the RR-interval sequence PSD within the so-called low frequency (LF) band (i.e., 0.04<frequency<0.15 Hz).

Many physiological variables are known to have a modulatory effect on the cardiac vagal efferents. In other words, many physiological factors can affect the heart rate control message sent to the heart via the efferent vagus nerves. For instance, the vagal efferent fibers are more powerfully excited during expiration by stimulating the arterial chemoreceptors and baroreceptors. Furthermore, it is indicated that the cardiac vagal preganglionic neurons may be directly modulated by central respiratory drive. Moreover, lung inflation itself may cause direct inhibition of cardiac vagal efferent activity. Finally, lung inflation is known to evoke tachycardia by stimulating the pulmonary C-fiber afferents. Thus, many physiological variables complexly interact to yield the final cardiac vagal efferent expression caused by respiration.

In some cases, even under quiescent resting supine conditions, heart rate changes may be highly variable and generally poorly reproducible. In addition to the previously mentioned cardiac vagal efferent modulators, the response of the heart itself may be a potential source of irreproducibility. The cardiac cycle, with its exquisitely orchestrated temporal/spatial arrangement of numerous physiological and molecular sub-systems is rich with phase dependencies. For instance, acetylcholine mediated (i.e., vagal mediated) regulation of sinus node automaticity may occur mainly during phase III repolarization; only during this brief phase is the cardiac cycle rate-sensitive to information arriving via the vagus nerve. Thus, to obtain enhanced reproducibility in these cases, it may be necessary to keep the cardiac cycle in phase with the cyclicity of the cardiac vagal efferents.

It may be demonstrated that spectral analysis of spontaneous RR-interval sequences results in spectral signatures that are characterized by an underlying 1/f noise. In other words, spontaneous recordings of RR-intervals results in power spectral densities that are inversely proportional to frequency. The net effect of the 1/f noise is the inability to accurately quantify the location and magnitude of important physiological spectral peaks, particularly those in the LF band (where the noise is greatest). A large component of this 1/f noise may be due to the summation of noise increments caused by the lack of temporal synchronization between the arrival of the cardiac vagal efferent messages and the timing of the short-lived cardiac phase III repolarization (which may establish the time to the next beat). For example, if the vagal efferent message arrives well before the cardiac phase III repolarization, then it may be likely that the sinus node neurotransmitter acetylcholine (ACh) will have decayed substantially before it can eventually affect the next cardiac phase III repolarization magnitude (and hence the time interval to the next beat). Time constants for sinus node ACh degradation kinetics for dogs and rabbits have been estimated at ˜500 ms, and thus it may be likely that that a significant proportion of sinus node ACh can be lost in the interbeat interval. As well, the sinus node ACh degradation kinetics may be shown to be exponential, thus the magnitude of ACh degradation is a nonlinear function of the time difference between the arrival of the vagal efferent message and the cardiac Phase III repolarization. This lack of consistent timing may add a random component to each R-R interval; the sum of these random increments may produce the 1/f type noise typical of spectra for spontaneous recordings.

### SUMMARY

There is therefore provided in one embodiment a method of controlling a cardiac cycle of a human being by detecting the cardiac cycle and intentionally coordinating a physiological activity with the cardiac cycle. The physiological activity, for example the respiratory cycle, is preferably matched to a point in the cardiac cycle such as the QRS of the cardiac cycle.

Operation of embodiments of the method may be enhanced using a heart monitor to measure the cardiac cycle and a device to measure the physiological activity, such as a respiratory cycle monitor. Coordination of the cycles may be enhanced by generating a signal coordinated with the cardiac cycle, or by monitoring synchronicity between the cardiac cycle and the physiological activity. Other physiological activity that may be coupled are a hand movement, whole body movement or response to an auditory or visual signal.

Cardiac coupled respiration may be useful to lower blood pressure, reduce nervousness as indicated by a quiverometer, lengthen the RR-interval (lower heart rate), allow relaxation in preparation for sleep and increased sport performance. Subjects may be provided with instruction sheets for carrying out the method steps, or may be instructed in a clinic setting.

A further method is disclosed. A physiological activity of a human being is coordinated with the cardiac cycle. A physiological variable for the human being is measured, the physiological variable having a characteristic response to the coordination of the cardiac cycle with the physiological activity. The measurement of the physiological variable is compared with a benchmark to determine if the human being has a normal or abnormal response.

In some embodiments, for example, the benchmark is a normal of the physiological variable derived from a class of human beings. In some embodiments, for example, the benchmark may be derived from longitudinal studies of one or more human beings, including the patient being tested.

A method of controlling a cardiac cycle of a human being is also disclosed. The cardiac cycle of a human being is detected. The human being then intentionally coordinates a physiological activity with the cardiac cycle to improve the physiological performance of the human being by at least one of lowering blood pressure, reducing nervousness, lowering heart rate, allowing relaxation, and increasing sport performance.

An apparatus for controlling a cardiac cycle of a human being is also disclosed. The apparatus comprises a cardiac cycle monitor, a sensor of a physiological activity, and a detector of coordination between the physiological activity and the cardiac cycle. The cardiac cycle monitor has a representation of the cardiac cycle as output. The sensor of a physiological activity has a representation of the physiological activity as output. The detector of coordination between the physiological activity and the cardiac cycle has the representation of the cardiac cycle and the representation of the physiological activity as input.

These and other aspects of the disclosed methods and apparatus are set out in the claims, which are incorporated here by reference.

### BRIEF DESCRIPTION OF THE FIGURES

Preferred embodiments of the disclosed methods and apparatus will now be described with reference to the figures, in which like reference characters denote like elements, by way of example, and in which:

FIG. 1 is a flow diagram showing the basic process steps and including a representation of the cardiac cycle of a human being;

FIG. 2 is a graph showing the effect of cardiac coupled respiration;

FIG. 3A is a graph showing synchronicity between a cardiac cycle and respiration of a human being carrying out cardiac coupled respiration;

FIG. 3B illustrates an embodiment of a method with feedback;

FIG. 4 shows two graphs indicating the effect of cardiac coupled respiration on blood pressure and RR-interval of a human being;

FIG. 5 shows effect on cardiac coupled respiration of addition of a muscle-heart reflex; and

FIG. 6 illustrates equipment useful for carrying out an embodiment of a phase-lock feedback loop.

FIG. 7 is a graph illustrates the effects of spontaneous and coherent respiration on blood pressure and the cardiac cycle.

FIG. 8 is a graph that illustrates the synchronization of the respiratory cycle with blood pressure and the cardiac cycle.

FIG. 9 is a graph that illustrates the coherence of the respiratory cycle with blood pressure and the cardiac cycle.

FIGS. 10-11 are sets of graphs that illustrate a comparison between the coordination of the respiratory cycle with the R-R intervals of healthy and diseased human beings.

FIG. 12 is a flow chart illustrating an embodiment of a method disclosed herein.

### DETAILED DESCRIPTION OF PREFERRED EMBODIMENTS

In the claims, the word “comprising” is used in its inclusive sense and does not exclude other elements being present. The indefinite article “a” before a claim feature does not exclude more than one of the feature being present.

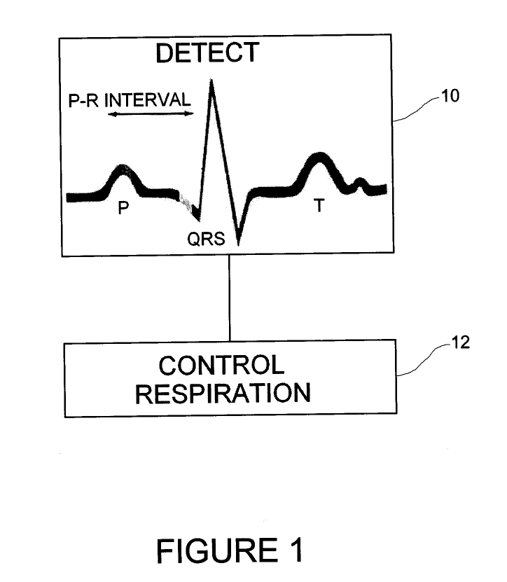

Referring to FIG. 1, in a first method step 10, the cardiac cycle is detected. As shown in FIG. 1, the electrical cardiac cycle of a human being follows a well established pathway from the P through the QRS to the T parts of the cycle. The P part of the cycle corresponds to depolarization of the atrium by the sinus node of the heart, and is followed after the P-R interval, typically about 0.2 seconds later, by the QRS, which corresponds to the depolarization of the ventricular. This cycle is controlled by activation of the sinus node, which causes a depolarization wave to spread through the heart and cause the heart muscles to contract and pump blood. The electrical cardiac cycle can be detected directly by a device such as an electrocardiogram. As the heart muscle contracts, the cardiac cycle may be detected by the human being as a pulsation, for example by placing pressure on an artery using a finger, or may be detected by such devices as a heart monitor. The RR interval in a cardiac cycle is the time between R parts of successive cycles.

Control of the cardiac cycle is effected by following the step 10 of detecting the cardiac cycle, as for example by the methods described in the immediately preceding paragraph, with the step 12 of intentionally coordinating a physiological activity with the cardiac cycle. An example of a physiological activity is the respiratory cycle of the human being. For example, the human being may control breathing to match a peak of inspiration or expiration with a point in the cardiac cycle. This makes the respiratory cycle in phase and synchronized with the cardiac cycle. Since the human respiration rate is much lower in frequency than the cardiac cycle, the frequency of the cardiac cycle should be a harmonic of the respiration frequency, as for example the fourth of fifth harmonic. For application of the method to other animals, other harmonics are applicable. The point in the cardiac cycle may be the QRS of the cardiac cycle for ease of detection.

Coordination of the cardiac cycle with for example breathing as a representative physiological activity may be readily carried out using an instrument such as a heart monitor to detect the cardiac signal, generating a visual or audible signal coordinated with the cardiac cycle and having the subject actively coordinate breathing with the visual or audible signal. For example, as the cardiac cycle reaches the QRS point, a signal may be given, thus enabling the subject to more readily match the point of maximum expiration or inspiration to the QRS.

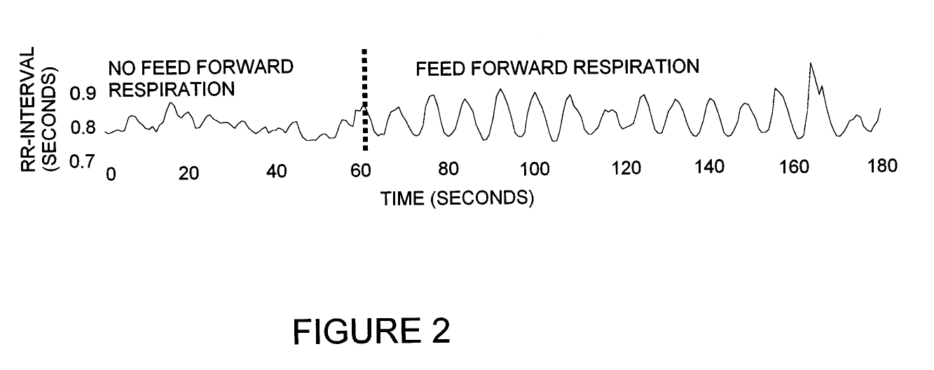

Response of the cardiac cycle to matching of breathing with the cardiac cycle is illustrated in FIG. 2. For the generation of FIG. 2, the subject rested quietly for 60 seconds, breathing spontaneously. The section of the graph indicated by “No Feed Forward Respiration” shows the irregular nature of the RR-interval sequence. After 60 seconds, the subject began feed forward respiration wherein end expiration was coupled to a QRS, and end inspiration was also coupled to a QRS. The subject repeatedly inhaled for 5 heart beats, and exhaled for 5 heart beats. As shown in FIG. 2, the RR-interval sequence quickly becomes periodic with a 0.1 Hz frequency.

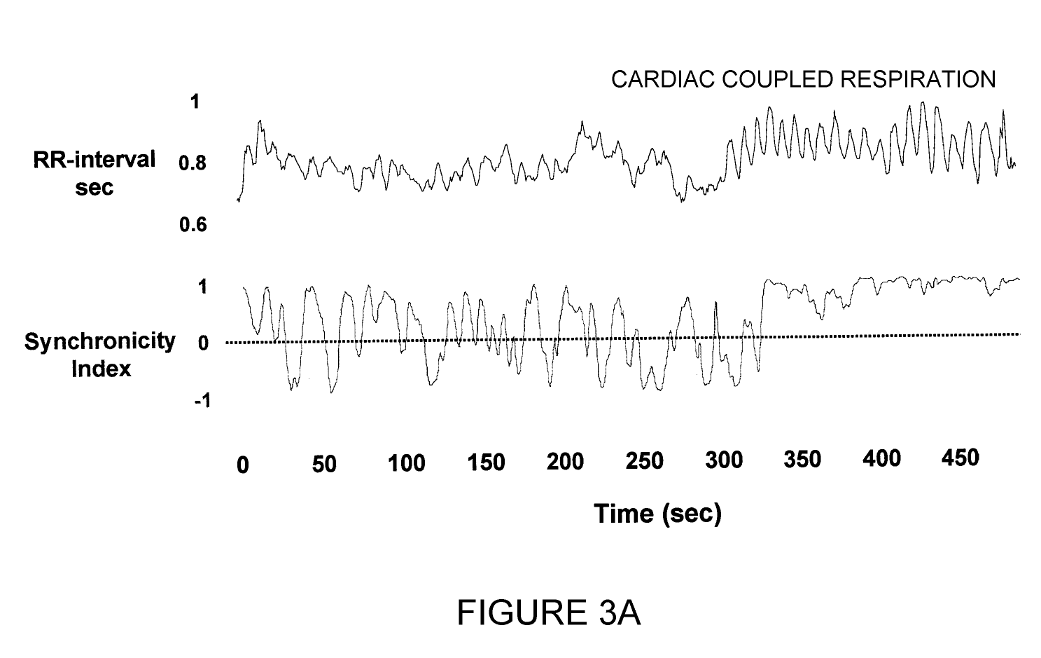

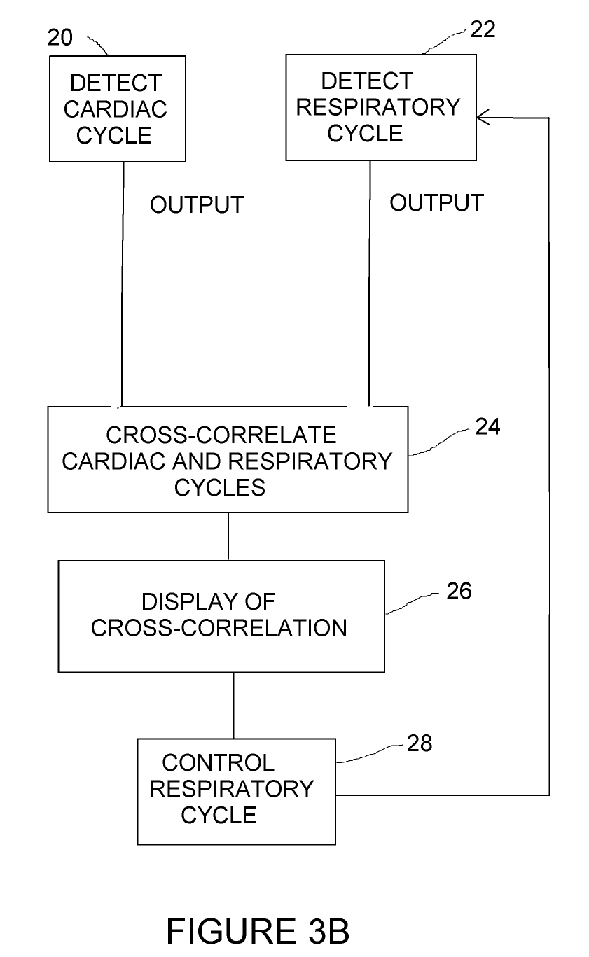

Coordination of the cardiac cycle with the physiological activity may be monitored to improve the coordination. Various methods may be used to measure the extent of coordination of the cardiac cycle with the physiological activity. For example, the respiratory cycle may be monitored using any of various devices such as a respiratory volume sensor. The respiratory volume sensor yields a time sequence of data whose value is an indication of respiratory volume. This time sequence of data may be correlated with a signal from a heart monitor to yield a synchronicity index. FIG. 3A shows a synchronicity index. The upper trace shows the RR-interval in seconds. The lower trace shows the synchronicity index found by a moving auto-correlation of the electric cardiac cycle. Synchronicity may also be found from cross-correlating respiratory volume with the cardiac cycle. For 300 seconds, the subject was at rest, with no cardiac coupled breathing. After 300 seconds, cardiac coupled respiration commenced, and the upper trace shows the evident greater periodicity of the RR-interval arising from cardiac coupled respiration. The lower trace shows the result of the cross-correlation. After 300 seconds, correlation values are near 1, indicating a high degree of coupling between the two signals. Improvement in the coupling may be obtained by varying the controlled variable, namely respiration, to achieve a high degree of synchronicity.

FIG. 3B illustrates the process steps. In step 20, the cardiac cycle is detected with for example a heart monitor. In step 22, the respiratory cycle is detected with for example a respiratory volume monitor. Output from the monitors is supplied to a processor, such as a computer, to cross-correlate the two outputs in step 24. A display, such as a visual or audible display, is used to display the cross-correlation in step 26. Various methods may be used to show the degree of cross-correlation such as the graph of FIG. 3A, or intensity, eg higher intensity meaning greater correlation, or sound, eg a louder sound indicating greater correlation. The subject may then vary the respiratory cycle, slowing or speeding up respiration, in step 28 using the displayed correlation to achieve a higher degree of correlation.

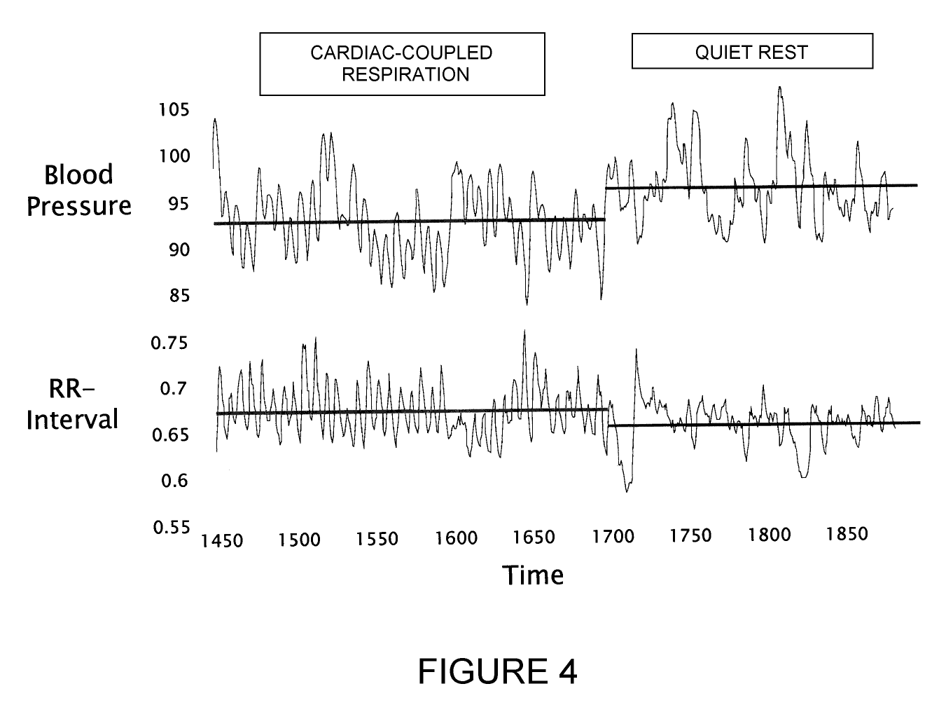

It has been found that cardiac coupled respiration, in at least some subjects, reduces blood pressure, increases the mean RR-interval and reduces nervousness as indicated by a quiverometer. The response of blood pressure, RR-interval, nervousness or other physiological parameter to the cardiac coupled respiration may be found by monitoring the respective physiological parameter with a suitable monitor, such as a blood pressure monitor, ECG, or quiverometer, respectively. A quiverometer is a device that measures involuntary muscle tremor, and may for example consist of a rod fixed to the end of a person's finger with an indicating point at the end of the rod, the location of which may be tracked by any suitable means. The monitor may be arranged to give a signal once a desired response of the physiological parameter has been achieved. FIG. 4 shows, upper trace, (A) reduction of blood pressure due to cardiac coupled respiration as compared with quiet rest and (B) increased RR-interval due to cardiac coupled respiration as compared with quiet rest. Lower blood pressure and increased RR-interval are generally considered to be beneficial for human beings, although the work underlying this patent document has not involved investigating improvements in health of subjects, other than measurement of these vital signs. Due to the increased RR-interval, reduced blood pressure and lower nervousness, cardiac coupled respiration is believed to be particularly useful when a subject is preparing for sleep, or carrying out a physical activity, such as a sport. Cardiac coupled respiration is believed to be particularly for a sport such as golf in which uniform repeated motions under low nervous activity are desirable.

It is believed that other physiological activities that cause a stimulus to the sinus node of the heart (muscle-heart reflex) will have a similar effect to respiration when coupled with the cardiac cycle. For example, physiological activities such as a hand movement, where a hand is caused to periodically grip and ungrip an object, or a whole body movement such as rocking a rocking chair, or an auditory or visual response to a repeated auditory or visual signal respectively are all believed to be capable of causing cardiac coupling.

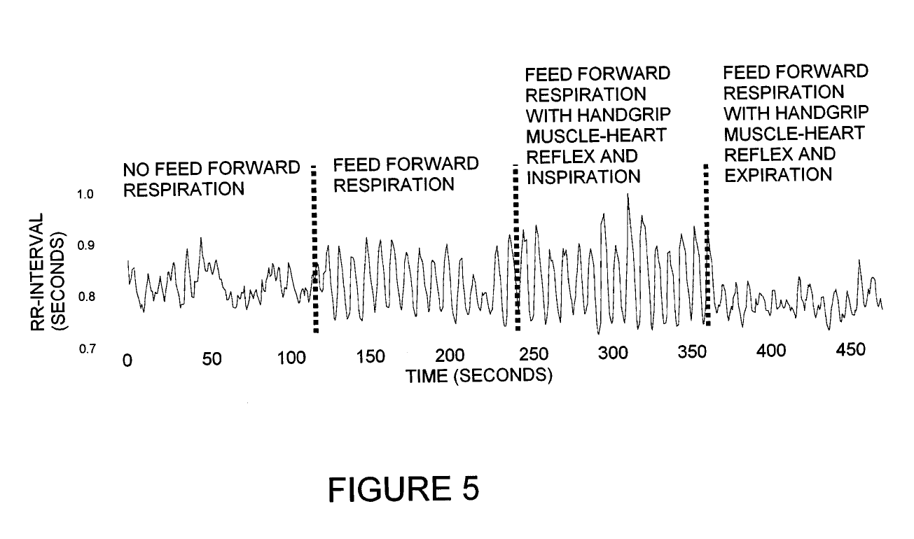

FIG. 5 illustrates the combined effects of feed forward respiration and muscle-heart reflex. For the first 60 seconds, the subject rested quietly breathing spontaneously. The RR-interval sequence is relatively irregular. After 120 seconds, the subject began feed forward respiration, where he repeatedly inhaled for 5 heart beats and exhaled for 5 heart beats. The RR-interval sequence quickly became periodic with a periodicity of ˜10 seconds, (I.e., frequency equal to ˜0.1 Hz). At 240 seconds, the subject began to squeeze a hand-grip for for the first 2 heart beats of the inhalation phase of respiration. The magnitude of the 0.1 Hz oscillations was increased. At 360 seconds, the subject began to squeeze a hand-grip for the first two beats of the expiration phase. The magnitude of the 0.1 oscillation was greatly reduced.

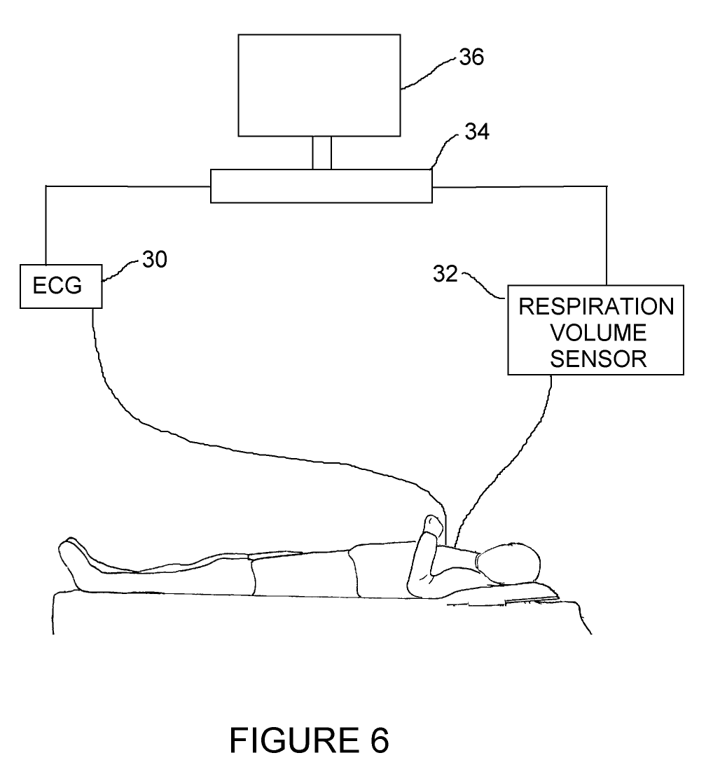

Referring to FIG. 6, various apparatus may be useful in effecting cardiac coupled physiological activity including a cardiac cycle monitor, such as an ECG apparatus 30, having a representation of the cardiac cycle as output. Also useful is a sensor of the physiological activity, having a representation of the physiological activity as output. A physiological sensor may also use a representation of the cardiac cycle as input, if the cardiac cycle also contains information about the physiological activity. For example, the cardiac cycle contains information about the respiratory cycle, and the sensor may be used to detect this information. A respiratory volume sensor 32 is also useful for the physiological activity sensor. To detect coordination between the physiological activity and the cardiac cycle, the outputs of the cardiac cycle monitor and the sensor may be input to a general purpose computer 34 and the two signals cross-correlated to yield the synchronization index of FIG. 3. The synchronization index may be viewed in real time on a computer monitor 36 to assist the subject in improving the coupling. By arranging for the cardiac cycle monitor to output a visual or audible signal at a recurring point in the cardiac cycle, the subject may more readily control the coupling of the physiological activity with the cardiac cycle.

Instructions sheets or other media carrying human readable instructions may be provided for instructing a person to carry out required method steps. For example, the media may direct a person to listen for an audible signal from a heart monitor that for example beeps at each 5th QRS and reach a point of deepest breathing in and then deepest breathing out at alternating beeps. Feedback for the subject may also be provided by directing the subject to monitor blood pressure and maintain a log to indicate whether there is any blood pressure reduction from following the process steps.

The control of the physiological activity that is being coordinated with the cardiac cycle is generally carried out intentionally by the subject, where the subject adverts to the cardiac cycle or a representation of the cardiac cycle, and deliberately controls the physiological activity. In the case of breathing, it is believed that coordination with the cardiac cycle may also be obtained by enforced regulation of the breathing cycle.

In some embodiments of the disclosed embodiments, a coherent state of dynamic cardio-respiratory equilibrium is achieved in humans. This provides the ability to acquire data under somatic conditions that are predictable and reproducible. A stable, reproducible dynamic condition is also necessary for the measurement of subtle physiological variables that are predictors of risk for sudden cardiac death. Hence, embodiments of the disclosed methods may be used in the assessment of cardiac vagal activity, assessment of coherent respo-baroreflex activity, and the enhanced ability to record T-wave alternans. These data acquisition methods may be referred to as coherent data acquisition, in the sense that the technology is used to establish a stable dynamic physiological equilibrium (i.e., periodic oscillations with coherent phase relationships between variables) of pertinent physiological variables. Then, sampling is carried out of the physiological variables as a function of the phase of the cyclic variables.

Despite the fact that some of the mechanisms responsible for the respiratory modulation of autonomic activity remain incompletely understood today, RSA magnitude may be widely used as a clinical measure of cardiac vagal activity. In the weeks immediately following acute myocardial infarction, RSA may be reduced, and within a year RSA may return to normal levels. Thus, normal RSA can be significantly diminished by medical trauma, only to be eventually re-established. Thus, there may be a need for simple risk tests for longitudinal assessment of patients.

The lack of consistent timing discussed in the background with regards to the 1/f noise in typical of spectra for spontaneous readings adds a “random” component to each RR-interval. The sum of these random increments may produce the 1/f type noise typical of spectra for spontaneous recordings. By using coherent data acquisition, the timing error may be purposefully minimalized and thus the timing-dependent random component may also be minimalized.

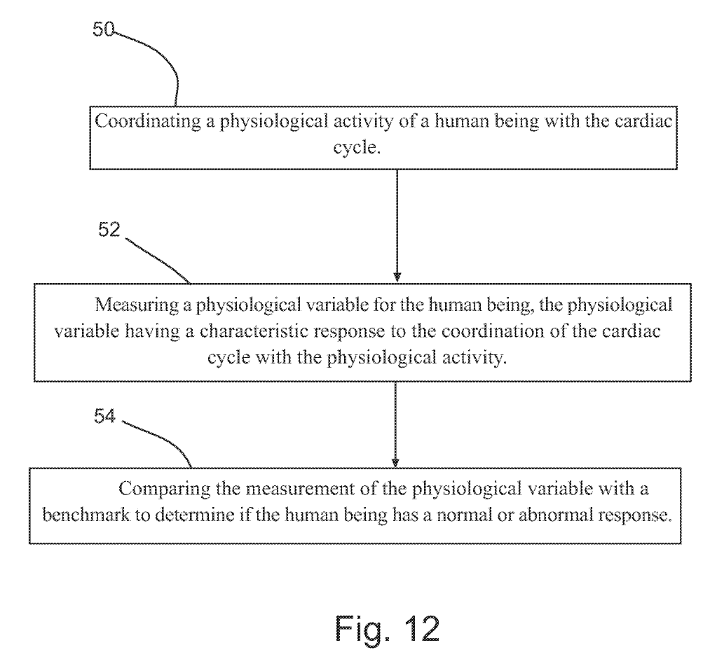

Referring to FIG. 12, a method is disclosed. In step 50, a physiological activity of a human being is coordinated with the cardiac cycle. The physiological activity may be the respiratory cycle of the human being. In one embodiment, coordinating further comprises establishing a breathing rate of between 0.05 and 0.17 Hz. In other embodiments, coordinating may further comprise establishing breathing rates in the LF band. Coordinating the physiological activity with the cardiac cycle may comprise, for example, matching a peak of inspiration or expiration with a point in the cardiac cycle. In some embodiments, the physiological activity is at least one of a hand movement, a whole body movement, and a response to an auditory or visual signal, for example. Coordinating may further comprise synchronizing.

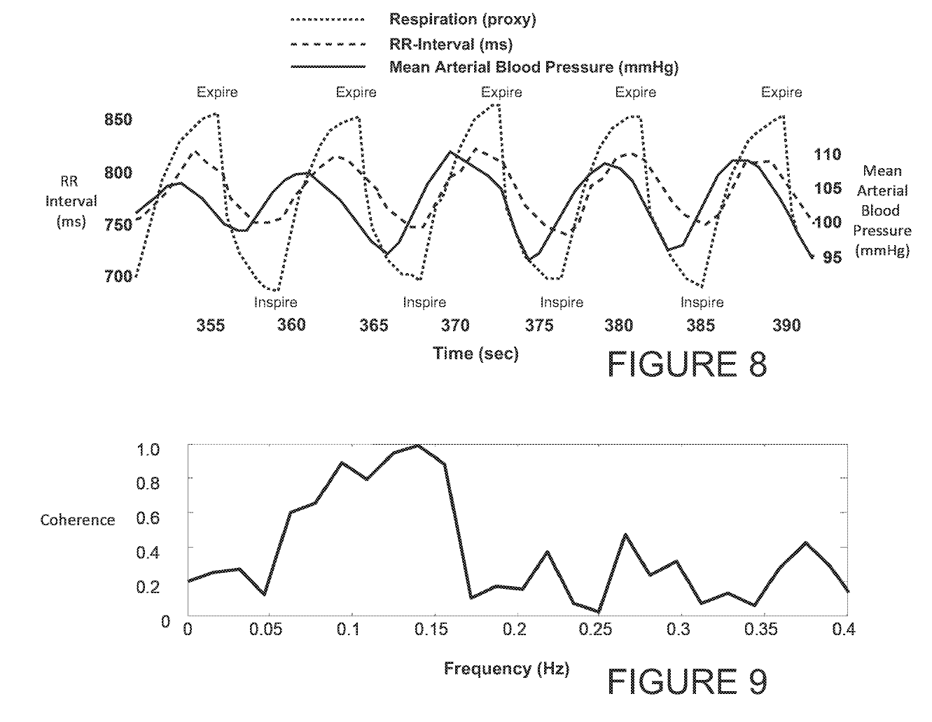

Referring to FIG. 12, in step 52, a physiological variable for the human being is measured, the physiological variable having a characteristic response to the coordination of the cardiac cycle with the physiological activity. Referring to FIG. 8, the physiological variable may be at least one of cardiac cycle, R-R interval, blood pressure, and breathing rate. Measuring may further comprise measuring at least one of the periodicity, frequency, magnitude, phase angle, and regularity of the physiological variable, for example. This is indicated in FIG. 8, as all of these characteristics may be measured from the information illustrated.

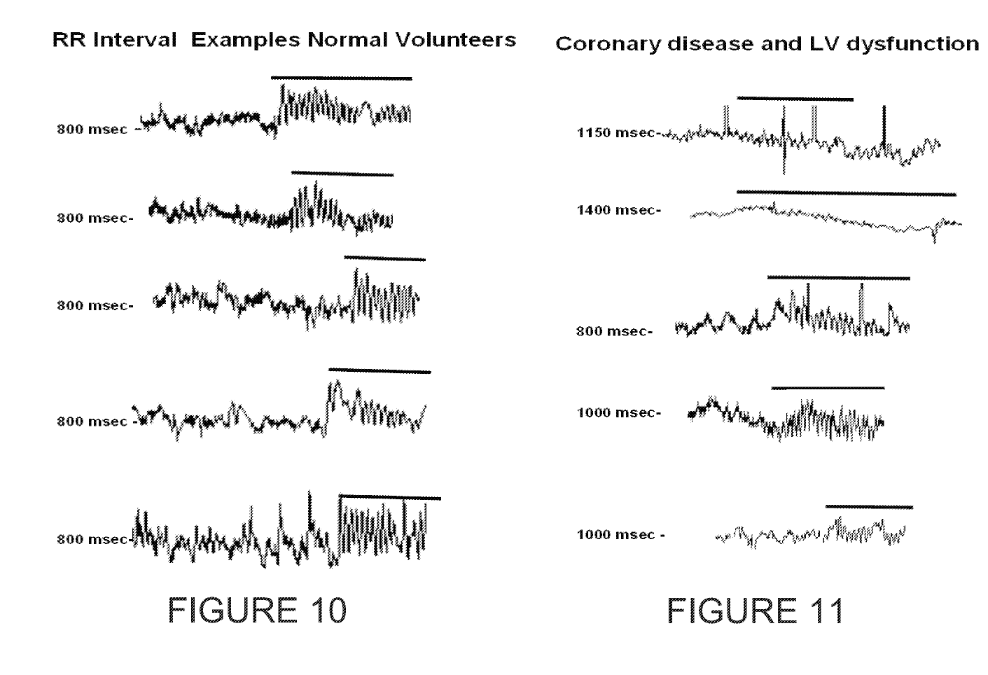

Referring to FIG. 12, in step 54, the measurement of the physiological variable is compared with a benchmark of the physiological variable to determine if the human being has a normal or abnormal response. In some embodiments, for example, the benchmark is a normal of the physiological variable derived from a class of human beings. In some embodiments, for example, the benchmark may be derived from longitudinal studies of one or more human beings, including the patient being tested. Referring to FIGS. 10 and 11, such a comparison may be carried out using, for example, the data from one of the patients from FIG. 11 (the diseased patients), and the normal of the data from the patients of FIG. 10. As is discussed in further detail below, there may be a correlation between, for example, the coherence of the phyisiological variable's response with the coordination in a diseased patient, and the same coherence in a set of normal patients. Thus, by measuring the physiological variable and comparing it with the benchmark, a determination on whether or not the human being has a normal or abnormal response may be carried out. This information may then be used in, for example, evaluating the risk for heart attack.

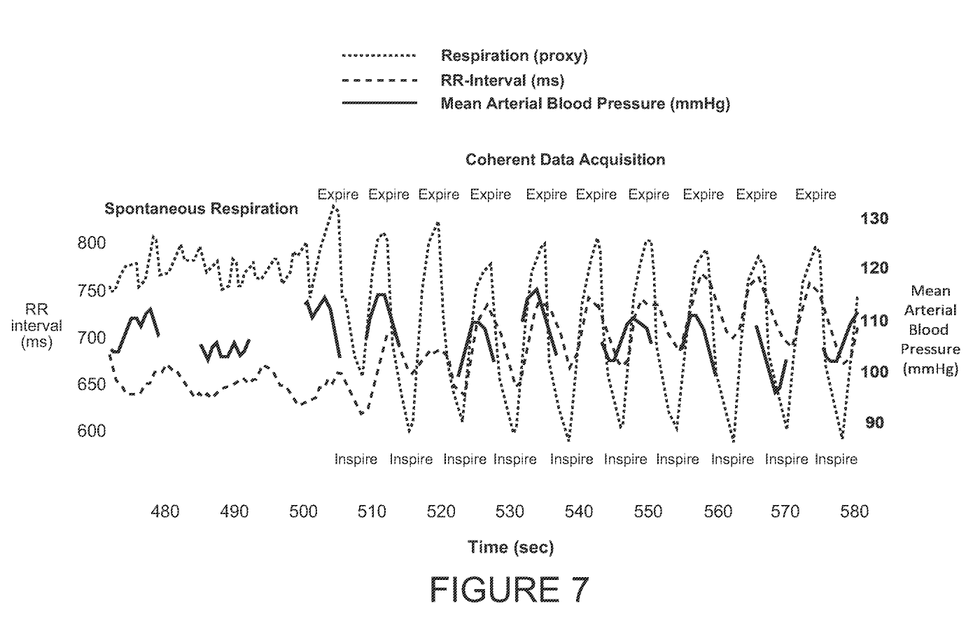

With knowledge of the potential sources of variance, a system to rhythmically synchronize the obvious physiological factors that can affect heart rate response via vagal mediated control has been developed. In one embodiment, given that RSA magnitude begins to attenuate at respiration frequencies >6 breaths per minute (bpm, i.e., 0.1 Hz), the synchronization protocol may be configured to yield respiration frequencies <6 bpm (0.1 Hz). RSA magnitude may be demonstrated as maximal at fixed respiration frequencies within the lower half of the LF band. This may be understandable given that power within the LF band may be directly attributed to baroreflex activity. Referring to FIG. 7, an example of spontaneous and coordinated respiration is illustrated. Before the 500 s mark, respiration is spontaneous and uncoordinated with the cardiac cycle. Accordingly, the RR interval and blood pressure remain relatively uncoordinated, driven by numerous feedback mechanisms. At the 500 s mark, the patient thus begins to coordinate their breathing pattern with the cardiac cycle. This may be carried out by any of the methods disclosed herein for example. For example, coordinating may further comprise measuring the cardiac cycle with an instrument and generating a signal coordinated with the cardiac cycle.

The transitions of breath pattern (ie inspire and expire) are labeled on the graph for reference. During, for example, fixed LF respiration, the magnitude of cyclic arterial blood pressure (BP) change may be significantly increased relative to BP changes occurring during spontaneous respiration, as illustrated. FIG. 7 illustrates that, relative to spontaneous respiration, the magnitude of the arterial blood pressure fluctuations increase greatly during coherent data acquisition. Referring to FIG. 8, BP changes may maintain, for example, a fixed phase relationship with respiration and the RR-interval changes, as revealed by a high spectral coherence illustrated in FIG. 9. In this manner, the coordination of the cardiac cycle with at least one of the physiological activity and the physiological variable may be synchronized. This synchronization may be in-phase, or partially out of phase. Partially out of phase synchronization may be the result of the delayed effects of the feedback mechanisms controlling the coordination. The synchronization does not have to be phase synchronized, provided that the various frequencies of the coordinated factors are substantially the same or harmonically related to one another. Referring to FIG. 8, attainment of synchronized cardio-respiratory dynamic equilibrium is thus illustrated. The relevant physiological variables may maintain consistent phase relationships at the driving respiratory frequency.

Referring to FIG. 9, spectral coherence is illustrated between mean arterial blood pressure and R-R interval obtained during coherent data acquisition. Thus, at respiratory frequencies within the LF band, there may be a strong coupling between, for example, BP, respiration and RR-interval change. Furthermore, the strong coherence between BP, RR-interval, and respiration, plus the continued cyclicity of BP and RR-interval indicate that autonomic control may have attained a state of dynamic equilibrium, wherein the continued LF respiration provides the cyclic forcing function that yields the ongoing cyclic autonomic response. In other words, the baroreflex may have a natural resonance frequency of ˜0.1 Hz. Thus, the fixed LF respiration (at ˜0.1 Hz) may provide enough appropriately timed driving force to keep the vascular/heart rate response oscillating at the baroreflex resonance frequency. When respiration frequency is reduced to 0.1 Hz, the measured baroreflex activity may actually be a hybrid metric: it combines the RR-interval changes due to direct and indirect vagal modulatory effects of respiration (i.e., RSA) with the RR-interval changes due to baroreflex activity. Despite this hybrid nature, the coherence between BP, respiration and RR-interval prevents this hybrid metric from being confounded by random respiration events (as happens when using the spontaneous method to determine baroreflex response function). This new hybrid measurement may be referred to as coherent respo-baro activity.

In one embodiment, the physiological variable may comprise the cardiac cycle, and the measuring step 54 may further comprise measuring the T-wave alternans of the cardiac cycle. This application of coherent acquisition relates to measurement of T-wave alternans. T-wave alternans are subtle (i.e., micro-volt) changes in the shape of the ECG complexes. Respiratory movements alter the electrical impedance of the chest cavity, and thus have an impact on the character (shape) of ECG signals. If respiration and the cardiac cycle are not synchronized, then this change in impedance due to respiration may add a significant random component of ECG voltage change that can confound the measurement of subtle T-wave alternans. In one application of this technology an environment of dynamic cardio-respiratory equilibrium may be created, for example through coherent data acquisition, wherein the cardiac cycle will no longer be randomly coupled to the respiration. Thus the morphology of the ECG cycles can be evaluated at fixed phase relationships to the respiratory cycle. For example, the shape of ECG complexes may be assessed at end expiration, and end expiration may be extended so that two successive complexes could be recorded to see if alternans (paired beat patterns) could be recorded under consistent dynamic environmental conditions.

In some embodiments, measuring further comprises measuring the extent of coordination of the physiological variable with the physiological activity. Measuring the physiological variable may further comprise measuring the coherence between the physiological variable and at least one of the cardiac cycle and the physiological activity. Referring to FIG. 7, if the physiological variable is blood pressure, for example, the coordination between the blood pressure and the respiratory cycle can be measured. The physiological variable may be, for example, at least one of the cardiac cycle, R-R interval, and blood pressure. In other embodiments, measuring further comprises measuring the extent of coordination of the physiological variable with the cardiac cycle. Referring to FIG. 7, this may also be observed, as the coordination between the physiological variable (for example blood pressure) and the cardiac cycle (for example the RR interval) can be measured. The physiological variable may be, for example, at least one of blood pressure and breathing rate.

Referring to FIGS. 10 and 11, to provide evidence that coherent data acquisition may allow recognition and measurement of coherent respo-baro activity, normal age matched subjects (N=18) were analyzed and compared to patients (N=23) with coronary heart disease and severe LV dysfunction. FIGS. 10 and 11 illustrate a comparison of RR-interval sequences from healthy aged-match control subjects with similar sequences from diseased subjects, respectively. The heavy overhead bars indicate application of embodiments of Coherent Data Acquisition technology. Non-barred sections of the sequence are derived from spontaneous respiration. The increased magnitude of coherent respo-baro activity (illustrated by the RR-intervals) for the healthy controls may be contrasted with the absence or reduction of coherent respo-baro activity in the diseased patients.

FIG. 11 shows a lack of clear baroreflex modulation of RR-interval under baseline (i.e., spontaneous) conditions, and a lack of the subsequent large magnitude periodic variation of RR-interval due to coherent respo-baro activity as illustrated in FIG. 10. Referring to FIG. 10, for all 18 healthy volunteers, the cardio-coupled respiration yielded well-organized cyclic RR-interval fluctuations, with measurements of the magnitude of the RR interval of coherent respo-baroreflex activity varying from 45 to 200 ms with a mean measurement of 125±58 ms. A representative example is shown in FIG. 8.

Referring to FIG. 11, of the 23 patients with coronary artery disease (CAD), heart failure and severe Left Ventricular (LV) dysfunction, 16 produced measureable coherent respo-baro activity during coherent data acquisition. For these, the magnitude of the RR interval of coherent respo-baro activity was generally small, 40±12 ms, as evidenced by, for example, the 3^{rd} and 4^{th} plots down in FIG. 11. In 7 other individuals, there was no measureable coherent respo-baro activity measured during coherent data acquisition. Thus, while all healthy subjects produced significant coherent respo-baro activity, many of the diseased patients did not, and for those diseased patients that did produce coherent respo-baro activity, the magnitude of such was significantly and measurably diminished. Thus, a lack of a significant coherent respo-baro activity may be associated with a poor heart function status.

In some embodiments, the method may comprise the steps of finding an indication of the extent of coordination of the cardiac cycle with the physiological activity, and using the indication of the extent of coordination to improve the coordination. In some embodiments, the method may further comprise the step of monitoring a physiological parameter that is responsive to the coordination of the cardiac cycle with the physiological parameter. Such methods may further comprise the step of producing a signal indicative of a desired response of the physiological parameter. In these embodiments, a patient may be aided in establishing, for example, optimal coordination. This may aid in, for example, the measuring step. Referring to FIG. 11, it can be seen that in some, particularly diseased, patients, it may be difficult to establish coherency, and thus these embodiments may be of value.

Immaterial modifications may be made to the embodiments of the methods and apparatus described here without departing from the invention.

What is claimed is:

1. A method comprising the steps of:
coordinating a physiological activity of a human being with the cardiac cycle;
measuring a physiological variable for the human being, the physiological variable having a characteristic response to the coordination of the cardiac cycle with the physiological activity; and
comparing the measurement of the physiological variable with a benchmark to determine if the human being has a normal or abnormal response.

2. The method of claim 1 in which the benchmark is a normal of the physiological variable derived from a class of human beings.

3. The method of claim 2 in which the physiological activity is the respiratory cycle of the human being.

4. The method of claim 3 in which the step of coordinating further comprises establishing a breathing rate of between 0.05 and 0.17 Hz.

5. The method of claim 2 in which the physiological variable comprises the cardiac cycle, and in which measuring further comprises measuring the T-wave alternans of the cardiac cycle.

6. The method of claim 2 in which measuring further comprises measuring at least one of the periodicity, frequency, magnitude, phase angle, and regularity of the physiological variable.

7. The method of claim 2 in which the physiological variable is at least one of cardiac cycle, R-R interval, blood pressure, and breathing rate.

8. The method of claim 2 in which measuring further comprises measuring the extent of coordination of the physiological variable with the physiological activity.

9. The method of claim 8 in which the physiological variable is at least one of the cardiac cycle, R-R interval, and blood pressure.

10. The method of claim 2 in which measuring further comprises measuring the extent of coordination of the physiological variable with the cardiac cycle.

11. The method of claim 10 in which the physiological variable is at least one of blood pressure and breathing rate.

12. The method of claim 2 in which coordinating the physiological activity with the cardiac cycle comprises matching a peak of inspiration or expiration with a point in the cardiac cycle.

13. The method of claim 2, in which coordinating further comprises measuring the cardiac cycle with an instrument and generating a signal coordinated with the cardiac cycle.

14. The method of claim 2 further comprising the steps of:
finding an indication of the extent of coordination of the cardiac cycle with the physiological activity; and
using the indication of the extent of coordination to improve the coordination.

15. The method of claim 2 further comprising the step of monitoring a physiological parameter that is responsive to the coordination of the cardiac cycle with the physiological parameter.

16. The method of claim 15 further comprising the step of producing a signal indicative of a desired response of the physiological parameter.

17. The method of claim 2 in which the physiological activity is at least one of a hand movement, a whole body movement, and a response to an auditory or visual signal.

18. The method of claim 2 in which measuring the physiological variable further comprises measuring the coherence between the physiological variable and at least one of the cardiac cycle and the physiological activity.

19. A method of controlling a cardiac cycle of a human being, the method comprising the steps of:
detecting the cardiac cycle of a human being; and
the human being intentionally coordinating a physiological activity with the cardiac cycle to improve the physiological performance of the human being by at least one of lowering blood pressure, reducing nervousness, lowering heart rate, allowing relaxation, and increasing sport performance.

20. Apparatus for controlling a cardiac cycle of a human being, the apparatus comprising:
a cardiac cycle monitor having a representation of the cardiac cycle as output;
a sensor of a physiological activity, having a representation of the physiological activity as output; and
a detector of coordination between the physiological activity and the cardiac cycle, the detector of coordination having the representation of the cardiac cycle and the representation of the physiological activity as input.
